(19) United States
(12) Patent Application Publication
(10) Pub. No.: US 2015/0359824 A1
(43) Pub. Date: Dec. 17, 2015

(54) IMPROVED OMEGA-3 FOOD SUPPLEMENT BASED ON MARINE OIL

(71) Applicant: Sami GUETARI, Bab Souika (TN)

(72) Inventor: Sami GUETARI, Bab Souika (TN)

(21) Appl. No.: 14/764,252

(22) PCT Filed: Jan. 22, 2014
(86) PCT No.: PCT/TN2014/000001

§ 371 Date: Jul. 29, 2015

(30) Foreign Application Priority Data
Jan. 29, 2013 (TN) 2013/0036

(51) Int. Cl. A61K 35/60; A61K 36/71; A61K 36/899; A61K 36/736; A23L 1/30; A61K 36/48; A61K 36/18; A61K 36/28; A61K 36/31; A61K 36/63; A61K 36/55

(52) U.S. Cl. CPC A61K 35/60; A61K 36/63; A61K 36/71; A61K 36/899; A61K 36/736; A61K 36/55; A61K 36/48; A61K 36/18; A61K 36/28; A61K 36/31; A23L 1/3006; A23V 2002/00

(57) ABSTRACT

A food supplement omega 3 based on marine oils (extracted from fish, fish byproducts, mollusks, krill, algae, microalgae) to fight against hypercholesterolemia and lipoprotein disruption leading to atherosclerosis and so to cardiovascular diseases. Optimization of Lipid Prevention Score (LPS) of at least one food supplement omega 3 by adding either high or low LPS oil (vegetable and/or marine) according to the LPS of the omega 3 based marine oils. This action permits to make a well balanced fatty acid product that will have a better effect against the rising of bad cholesterol levels in the blood. This product can be prescribed as part of a preventive diet and/or curative treatment for cardiovascular diseases since such illnesses lead to the highest human mortality in the industrialized countries (about 180,000 deaths annually in France, which corresponds to 32% of total deaths).

The present invention is about the non-therapeutic use of at least one food supplement based on marine oils (fish, fish byproducts, mollusks, krill, algae, microalgae) enriched with vegetable oil and/or marine oil, as active ingredient in a food supplement or a para-pharmaceutical product.

Omegas 3 are poly-unsaturated fatty acids essential for the human body proper functioning, which can synthesize them only in extremely low quantities. Food is the main source of supply. They are commonly formulated in a liquid and oily state. At equal number of carbon, they have lower melting point compared to saturated fatty acids.

The monounsaturated fatty acids belonging to the families (n-7) and (n-9) such as palmitoleic acid C16: 1 (n-7) and oleic acid C18: 1 (n-9) very present in olive oil, act as precursor of other polyunsaturated fatty acids.

The alpha-linolenic acid: C18: 3 n-3 and linoleic acid: C18: 2 n-6, are called essential fatty acids, they are metabolized in order to synthesize polyunsaturated fatty acids (PUFAs).

The market for omega-3 is still growing both in terms of food and beverages as well as food supplements. In 2010, the market of foods and beverages with improved omega-3 content represented $4 billion against $1.3 billion for food supplements. According to the center of market analysis Packaged Facts, it is expected to grow by 15-20% during this decade.

Omegas 3 are used in Agro-Food Industry which introduce regularly new products enriched in EPA and DHA (yogurts, milks, eggs, and margarines etc.) to meet the consumer demand, looking for a better health and well being.

These products are made from marine oils (fish, byproducts of fish, krill, algae, microalgae). The question is to check whether the composition of these marine oils is well balanced in terms of fatty acids?

To tackle the problem, we used an indicator: the Lipid Prevention Score (LPS) which is an indicator calculated for every food. It takes into account total lipid content (L) and its composition of SFA, MUFA and PUFA. It allows to classify and to choose food for better prevention of cardiovascular diseases. LPS is highly and significantly correlated with the parameters contained in fatty foods.

LPS is calculated by applying the following formula:

LPS=L+2 SFA-1,184 MUFA-0,4 PUFA

This equation shows that if the proportions SFA, MUFA and PUFA of a given food are respectively equal to 33%, 50% and 17%, LPS is equal to L and the food is considered properly balanced in terms of fatty acids. If the proportion of SFA exceeds 33%, LPS is higher than L, while if the MUFA or PUFA exceed 50% or 17% respectively, LPS is less than L. therefore we have to compensate the foods with high LPS, by foods with low LPS.

For example, we are going to study the lipid composition of the oils extracted from the byproducts of fattened bluefin tuna, mainly the oils extracted from mesentery, liver, swim bladder, stomach, heart and kidneys.

### EXAMPLE 1

### The Fatty Acids of the Mesentery

The amount of total fatty acids present in the mesentery of farmed bluefin tuna is equal to 43.25 g /100 g FM (Table 1).

The saturated fatty acids constitute 32.02% of this amount. The mono unsaturated fatty acids constitute 29.57%. The poly unsaturated fatty acids (excluding EPA and DHA) constitute 9.5%. The EPA constitutes 14.17% and the DHA 7.91% (FIG. 1).

The report IFA/SFA is equal to 1.91. The report PUFA/SFA equals 0.98. The ratio of n-3/n-6 is equal to 11.85 and the ratio n-6/n-3 is equal to 0.084.

TABLE 1
Fatty acid composition of the mesentery the blue fin tuna fattened.

| No | RT | FA | % |
|---|---|---|---|
| 1 | 4.02 | C14:0 | 7.347 |
| 2 | 4.36 | NI | 0.29 |
| 3 | 4.68 | NI | 0.74 |
| 4 | 5.47 | C16:0 | 20.32 |
| 5 | 5.66 | C16:1n-7 | 8.46 |
| 6 | 5.95 | NI | 0.51 |
| 7 | 6.17 | NI | 1.07 |
| 8 | 6.26 | NI | 0.28 |
| 9 | 6.61 | C16:4 n-1 | 0.33 |
| 10 | 7.08 | C18:0 | 1.28 |
| 11 | 7.61 | C18:0 | 4.37 |
| 12 | 7.82 | C18:1 n-9 | 13.7 |
| 13 | 7.89 | C18:1 n-7 | 3.27 |
| 14 | 8.22 | C18:2 n-4 | 0.39 |
| 15 | 8.40 | C18:2 n-6 | 1.06 |
| 16 | 8.70 | C18:3 n-6 | 0.53 |
| 17 | 9.12 | NI | 0.29 |
| 18 | 9.34 | C18:3 n-3 | 0.78 |
| 19 | 9.81 | C18:4 n-3 | 2.15 |
| 20 | 10.01 | C18:5 n-3 | 0.37 |
| 21 | 10.81 | NI | 0.56 |
| 22 | 11.09 | C20:1 n-9 | 2.48 |
| 23 | 11.58 | NI | 0.42 |
| 24 | 12.84 | C20:4 n-3 | 0.95 |
| 25 | 13.77 | C20:4 n-6 | 0.81 |
| 26 | 14.24 | C20:5 n-3 | 14.17 |
| 27 | 15.37 | C22:1 n-11 | 1.64 |
| 29 | 19.43 | C22:5 n-3 | 2.13 |
| 30 | 20.14 | C22:6 n-3 | 7.91 |
| [symbol] NI | — | — | 4.05 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### EXAMPLE 2

### The Fatty Acids of the Liver

The average amount of total fatty acids present in the liver of farmed blue fin tuna is equal to 13 g /100 g FM (Table 2).

The saturated fatty acids constitute 32.18% of this amount. The mono unsaturated fatty acids constitute 34.38%. The poly unsaturated fatty acids (excluding EPA and DHA) constitute 9.53%. The EPA constitutes 12.82% and the DHA 6.6% (FIG. 2).

The report IFA/SFA is equal to 1.96. The report PUFA/SFA equals 0.90. The ratio of n-3/n-6 is equal to 10.31 and the ratio n-6/n-3 is equal to 0.096.

TABLE 2
Fatty acid composition of the blue fin tuna fattened liver.

| No | RT | FA | % |
|---|---|---|---|
| 1 | 4.02 | C14:0 | 3.64 |
| 2 | 4.36 | NI | 0.13 |
| 3 | 4.68 | NI | 0.43 |
| 4 | 5.08 | NI | 0.18 |
| 5 | 5.49 | C16:0 | 20.87 |
| 6 | 5.67 | C16:1 n-7 | 6.52 |
| 7 | 5.95 | NI | 0.55 |
| 8 | 6.17 | NI | 0.59 |
| 9 | 6.64 | C16:4 n-1 | 0.52 |
| 10 | 7.09 | C18:0 | 7.67 |
| 11 | 7.65 | C18:0 | 7.67 |
| 12 | 7.87 | C18:1 n-9 | 19.24 |
| 13 | 7.94 | C18:1 n-7 | 4.16 |
| 14 | 8.24 | C18:2 n-4 | 0.49 |
| 15 | 8.42 | C18:2 n-6 | 0.81 |
| 16 | 8.71 | C18:3 n-6 | 0.73 |
| 17 | 9.13 | NI | 0.38 |
| 18 | 9.35 | C18:3 n-3 | 0.44 |
| 19 | 9.82 | C18:4 n-3 | 1.07 |
| 20 | 10.01 | C18:5 n-3 | 0.31 |
| 21 | 10.83 | NI | 0.26 |
| 22 | 11.09 | C20:1 n-9 | 2.76 |
| 23 | 11.59 | NI | 0.72 |
| 24 | 12.84 | C20:4 n-3 | 0.86 |
| 25 | 13.78 | C20:4 n-6 | 0.93 |
| 26 | 14.28 | C20:5 n-3 | 12.82 |
| 27 | 15.39 | C22:1 n-11 | 1.70 |
| 28 | 16.85 | NI | 0.828 |
| 29 | 19.43 | C22:5 n-3 | 3.37 |
| 30 | 20.17 | C22:6 n-3 | 6.60 |
| [symbol] NI | — | — | 4.06 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### EXAMPLE 3

### The Fatty Acids of the Swim Bladder

The amount of total fatty acids present in the swim bladder of farmed blue fin tuna is equal to 28.5 g /100 g FM (Table 3).

The saturated fatty acids constitute 32.45% of this amount. The mono unsaturated fatty acids constitute 28.45%. The poly unsaturated fatty acids (excluding EPA and DHA) constitute 9.6%. The EPA constitutes 13.57% and the DHA 6.6% (FIG. 3).

The report IFA/SFA is equal to 1.86. The report PUFA/SFA is equals to 0.98. The ratio n-3/n-6 is equal to 10.56 and the ratio n-6/n-3 is equal to 0.094.

TABLE 3
Fatty acid composition of the swim bladder
of the blue fin tuna fattened.

| No | RT | FA | % |
|---|---|---|---|
| 1 | 4.03 | C14:0 | 8.50 |
| 2 | 4.36 | NI | 0.74 |
| 3 | 4.69 | NI | 0.71 |
| 4 | 5.48 | NI | 19.35 |
| 5 | 5.67 | C16:0 | 8.17 |
| 6 | 5.95 | C16:1 n-7 | 0.47 |
| 7 | 6.17 | NI | 1.05 |
| 8 | 6.26 | NI | 0.25 |
| 9 | 6.62 | C16:4 n-1 | 0.27 |
| 10 | 7.08 | C18:0 | 1.24 |
| 11 | 7.62 | C18:0 | 4.60 |
| 12 | 7.82 | C18:1 n-9 | 12.39 |
| 13 | 7.88 | C18:1 n-7 | 4.04 |
| 14 | 8.22 | C18:2 n-4 | 0.19 |
| 15 | 8.40 | C18:2 n-6 | 0.95 |
| 16 | 8.71 | C18:3 n-6 | 0.51 |
| 17 | 9.12 | NI | 0.54 |
| 18 | 9.34 | C18:3 n-3 | 0.97 |
| 19 | 9.81 | C18:4 n-3 | 2.05 |
| 20 | 10.01 | C18:5 n-3 | 0.32 |
| 21 | 10.82 | NI | 0.28 |
| 22 | 11.08 | C20:1 n-9 | 2.42 |
| 23 | 11.57 | NI | 0.39 |
| 24 | 12.83 | C20:4 n-3 | 1.02 |
| 25 | 13.78 | C20:4 n-6 | 1.27 |
| 26 | 14.24 | C20:5 n-3 | 13.57 |
| 27 | 15.39 | C22:1 n-11 | 1.43 |
| 28 | 16.86 | NI | 1.34 |
| 29 | 19.43 | C22:5 n-3 | 2.05 |
| 30 | 20.15 | C22:6 n-3 | 8.86 |
| [symbol] NI | — | — | 5.77 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### EXAMPLE 4

### The Heart of Fatty Acids

The amount of total fatty acids present in the heart of farmed bluefin tuna is equal to 1.65 g /100 g FM (Table 4).

The saturated fatty acids constitute 36.27% of this amount. The mono unsaturated fatty acids constitute 26.9%. The poly unsaturated fatty acids (excluding EPA and DHA) constitute 8.06%. The EPA constitutes 10.82% and the DHA 14.97% (FIG. 4).

The report IFA/SFA is equal to 1.67. The report PUFA/SFA equals 0.93. The ratio of n-3/n-6 is equal to 17.88 and the ratio n-6/n-3 is equal to 0.055.

TABLE 4
Fatty acid composition of heart of blue fin tuna fattened.

| No | RT | FA | % |
|---|---|---|---|
| 1 | 4.03 | C14:0 | 3.83 |
| 2 | 4.37 | NI | 0.16 |
| 3 | 4.69 | NI | 0.47 |
| 4 | 5.09 | NI | 0.16 |
| 5 | 5.50 | C16:0 | 22.56 |
| 6 | 5.68 | C16:1 n-7 | 5.22 |
| 7 | 5.96 | NI | 0.45 |
| 8 | 6.18 | NI | 0.55 |
| 9 | 6.71 | C16:4 n-1 | 0.38 |
| 10 | 7.10 | C18:0 | 3.64 |
| 11 | 7.66 | C18:0 | 9.88 |
| 12 | 7.86 | C18:1 n-9 | 12.12 |
| 13 | 7.91 | C18:1 n-7 | 4.62 |
| 14 | 8.25 | C18:2 n-4 | 0.23 |
| 15 | 8.43 | C18:2 n-6 | 0.77 |
| 16 | 8.72 | C18:3 n-6 | 0.43 |
| 17 | 9.10 | NI | 0.47 |
| 18 | 9.35 | C18:3 n-3 | 0.47 |
| 19 | 9.82 | C18:4 n-3 | 0.83 |
| 20 | 10.02 | C18:5 n-3 | 0.15 |
| 22 | 11.09 | C20:1 n-9 | 2.25 |
| 23 | 11.57 | NI | 0.89 |
| 24 | 12.83 | C20:4 n-3 | 2.07 |
| 25 | 13.77 | C20:4 n-6 | 0.56 |
| 26 | 14.27 | C20:5 n-3 | 10.82 |
| 27 | 15.37 | C22:1 n-11 | 1.67 |
| 29 | 19.43 | C22:5 n-3 | 2.17 |
| 30 | 20.19 | C22:6 n-3 | 14.97 |
| [symbol] NI | — | — | 3.65 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### EXAMPLE 5

### The Fatty Acids of the Stomach

The amount of total fatty acids present in the stomach of farmed bluefin tuna is equal to 2.65 g /100 g FM (Table 5). The saturated fatty acids constitute 36.13% of this amount. The mono unsaturated fatty acids constitute 22.03%. The poly unsaturated fatty acids (excluding EPA and DHA) constitute 11.15%. The EPA constitutes 13.68% and the DHA 12.5% (FIG. 5).

The report IFA/SFA is equal to 1.64. The report PUFA/SFA equals 1.13. The ratio of n-3/n-6 is equal to 18.83 and the ratio n-6/n-3 is equal to 0.053.

TABLE 5
Fatty acid composition of the stomach the blue fin tuna fattened.

| No | RT | FA | % |
|---|---|---|---|
| 1 | 4.02 | C14:0 | 4.2 |
| 2 | 4.28 | NI | 0.82 |
| 3 | 4.69 | NI | 0.80 |
| 4 | 5.08 | NI | 0.34 |
| 5 | 5.47 | C16:0 | 21.64 |
| 6 | 5.66 | C16:1 n-7 | 4.54 |
| 7 | 5.97 | NI | 0.20 |
| 8 | 6.17 | NI | 0.35 |
| 9 | 6.74 | C16:4 n-1 | 0.29 |
| 10 | 7.09 | C18:0 | 0.34 |
| 11 | 7.62 | C18:0 | 10.26 |
| 12 | 7.82 | C18:1 n-9 | 11.25 |
| 13 | 7.88 | C18:1 n-7 | 4.02 |
| 14 | 8.22 | C18:2 n-4 | 0.14 |
| 15 | 8.40 | C18:2 n-6 | 0.91 |
| 16 | 8.70 | C18:3 n-6 | 0.41 |
| 17 | 9.10 | NI | 0.40 |
| 18 | 9.35 | C18:3 n-3 | 0.49 |
| 19 | 9.81 | C18:4 n-3 | 0.75 |
| 20 | 10.01 | C18:5 n-3 | 0.29 |
| 21 | 10.81 | NI | 0.22 |
| 22 | 11.09 | C20:1 n-9 | 1.56 |
| 23 | 11.57 | NI | 0.24 |
| 24 | 12.83 | C20:4 n-3 | 4.77 |
| 25 | 13.77 | C20:4 n-6 | 0.54 |
| 26 | 14.24 | C20:5 n-3 | 13.68 |
| 27 | 15.38 | C22:1 n-11 | 0.66 |
| 28 | 16.84 | NI | 0.64 |
| 29 | 19.41 | C22:5 n-3 | 2.56 |
| 30 | 20.13 | C22:6 n-3 | 12.50 |
| [symbol] NI | — | — | 4.35 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### EXAMPLE 6

### The Fatty Acids of the Kidney

The amount of total fatty acids present in the kidney of farmed bluefin tuna is equal to 0.94 g /100 g FM (Table 6).

The saturated fatty acids constitute 29.32% of this amount. The mono unsaturated fatty acids constitute 32.39%. The poly unsaturated fatty acids (excluding EPA and DHA) constitute 11.15%. The EPA constitutes 11.71% and the DHA 8.86% (FIG. 5).

The report PFA/SFA is equal to 2.17. The report PUFA/SFA equals 1.07. The ratio of n-3/n-6 is equal to 13.1 and the ratio n-6/n-3 is equal to 0.076.

TABLE 6
Fatty acid composition of kidney blue fin tuna fattened.

| No | TR | AG | % |
|---|---|---|---|
| 1 | 4.03 | C14:0 | 4.54 |
| 2 | 4.36 | NI | 0.41 |
| 3 | 4.69 | NI | 0.51 |
| 4 | 5.08 | NI | 0.05 |
| 5 | 5.48 | C16:0 | 18.73 |
| 6 | 5.67 | C16:1 n-7 | 6.60 |
| 7 | 5.95 | NI | 0.44 |
| 8 | 6.17 | NI | 0.71 |
| 9 | 6.63 | C16:4 n-1 | 0.43 |
| 10 | 7.09 | C18:0 | 0.59 |
| 11 | 7.63 | C18:0 | 6.05 |
| 12 | 7.84 | C18:1 n-9 | 15.37 |
| 13 | 7.89 | C18:1 n-7 | 4.66 |
| 14 | 8.23 | C18:2 n-4 | 0.33 |
| 15 | 8.41 | C18:2 n-6 | 0.84 |
| 16 | 8.72 | C18:3 n-6 | 0.51 |
| 17 | 9.13 | NI | 0.32 |
| 18 | 9.30 | C18:3 n-3 | 0.64 |
| 19 | 9.82 | C18:4 n-3 | 1.32 |
| 20 | 10.02 | C18:5 n-3 | 0.33 |
| 21 | 10.83 | NI | 0.59 |
| 22 | 11.10 | C20:1 n-9 | 3.81 |
| 23 | 11.60 | NI | 0.89 |
| 24 | 12.85 | C20:4 n-3 | 0.88 |
| 25 | 13.78 | C20:4 n-6 | 0.86 |
| 26 | 14.26 | C20:5 n-3 | 11.37 |
| 27 | 15.39 | C22:1 n-11 | 1.95 |
| 28 | 16.85 | NI | 1.69 |
| 29 | 19.44 | C22:5 n-3 | 5.57 |
| 30 | 20.16 | C22:6 n-3 | 8.86 |
| [symbol] NI | — | — | 5.61 |

(No: peak number; RT: retention time; FA: undeveloped formula of the fatty acid; %: average percentage of the fatty acid in question compared to TFA; NI: unidentified fatty acid; [symbol] NI: Sum of % means of unidentified fatty acids; TFA: % of total fatty acids present in 100 g of fresh material).

### SUMMARY AND CONCLUSION

Using the fatty acids compositions of these six examples, we calculated the

Lipid Prevention Score for each of the extracted oils. The results are shown in Table (7).

TABLE 7
LPS of the oils extracted from the
byproducts of fattened bluefin tuna

| 100 g oil of | L | LSP |
|---|---|---|
| Mesentery | 100 | 114.7 |
| Swim bladder | 100 | 116.1 |
| Liver | 100 | 110.4 |
| Stomach | 100 | 129.5 |
| Heart | 100 | 125.7 |
| Kidney | 100 | 105.2 |

According to these results, LPS of the extracted oils from the byproducts of fattened bluefin tuna are higher than the total fat (L). Therefore it has to be adjusted by the addition of other foods with low LPS.

Consequently, we thought about the Tunisian olive oil. The proportions of the different types of fatty acids in seven different local olive varieties were studied by Dhifi in 2005 (Table 8) and reported below:

TABLE 8
The proportions of the different types of fatty acids present in
the oil extracted from seven Tunisian olive varieties.

| | (100 g of olive oil/variety) | | | | | | |
|---|---|---|---|---|---|---|---|
| | Chetoui | Chemlali | Chemchali | Sayali | Meski | Zalmati | Oueslati |
| SFA (%) | 9.3 | 20.1 | 16.9 | 13.1 | 14.1 | 20.0 | 14.1 |
| MUFA (%) | 71.2 | 58.9 | 62.9 | 81.4 | 61.2 | 59.4 | 61.2 |
| PUFA (%) | 19.5 | 21.2 | 20.2 | 5.8 | 24.7 | 20.5 | 24.7 |

Using these data, we calculated the LPS for each oil and the results are shown in Table 9:

TABLE 9
The Lipid Prevention Score for the different varieties of
Tunisian olive oil.

| | (100 g of olive oil variety) | | | | | | |
|---|---|---|---|---|---|---|---|
| | Chetoui | Chemlali | Chemchali | Sayali | Meski | Zalmati | Oueslati |
| LSP | 26.5 | 62 | 51.2 | 27.5 | 45.8 | 61.4 | 45.8 |
| L | 100 | 100 | 100 | 100 | 100 | 100 | 100 |

The values reported in table (9) show that LPS of all the studied olive oils are lower enough with respect to the total fat contained in these oils. They represent in fact foods with low LPS and could be used to adjust the high LPS of the oils extracted from the byproducts of fattened bluefin tuna.

According to Dhifi (2005), the local variety Chemlali is almost the most common in the regions of Centre, Sahel and South of Tunisia. The yield of oil is about 20 to 22%. This high yield is related to the soil texture which allows the olive trees to develop a huge rooting system. For these reasons, we choose the oil of this variety to adjust the high LPS of the oils extracted from the byproducts of fattened bluefin tuna.

In conclusion, in order to produce a food supplement with fatty acids' content properly balanced, with an optimized Lipid Prevention Score; 1 g of the final product (food supplement based on oil extracted from the byproducts of fattened bluefin tuna) has to include a determined quantity of olive oil (Chemlali variety) as reported in Table 10:

TABLE 10
olive oil proportion (Chemlali variety) that
should be included in 1 g of final product.

| | Mes- | Swim | | | | |
|---|---|---|---|---|---|---|
| Byproducts oils | entery | blasser | Liver | Stomach | Heart | Kidney |
| % of olive oil | 23.7 | 26 | 16.7 | 47.6 | 41.4 | 8.3 |
| (Chemlali variety) | | | | | | |

It is important to highlight that, this invention is not specific to the mixture of olive oil with oils extracted from the byproducts of fattened bluefin tuna. It is possible to use different types of marine oils (fish, fish byproducts, mollusks, mollusks byproducts, krill, algae, microalgae) and mix them with any kind of vegetable oil and/or marine oil (with low and/or high Lipid Prevention Score), with specific proportions in order to have a final product with optimum Lipid Prevention Score, properly balanced on its fatty acids, that can be prescribed as part of a preventive diet and/or curative treatment to fight against hypercholesterolemia and lipoprotein disruption which lead to atherosclerosis and so to cardiovascular diseases.

For example, Table No. 11 specifies the fatty acids composition and the Lipid Prevention Scores of some vegetable and marine oils.

TABLE 11
Fatty acids composition (% of total fatty acids) and Lipid
Prevention Scores of some vegetable and marine oils

| | SFA | MUFA | PUFA | LPS |
|---|---|---|---|---|
| Oil Nigella (black cumin) | 11.93 | 23 | 56.2 | 74.1 |
| corn oil | 13.5 | 28.9 | 57.6 | 69.7 |
| sweet almond oil | 8 | 70 | 17 | 26.3 |
| linseed oil | 9 | 20 | 66 | 67.9 |
| Peanut oil | 21 | 57 | 22 | 65.7 |
| hazelnut oil | 7 | 78 | 10 | 17.6 |
| sunflower oil | 14 | 22 | 64 | 76.3 |
| olive oil | 12 | 81 | 7 | 25.9 |
| canola oil | 6 | 56 | 33 | 32.4 |
| nut oil | 9 | 23 | 63 | 65.5 |
| Krill Oil | 28.5 | 19.5 | 27.5 | 122.9 |
| Mackerel oil | 30.5 | 25.5 | 40.5 | 114.6 |
| Sardines oil | 40.5 | 37.1 | 45.5 | 118.9 |
| anchovy oil | 34 | 37.5 | 61.5 | 99.0 |
| Oil squid | 24 | 49.5 | 62 | 64.6 |
| Cod liver oil | 22.6 | 46.71 | 22.54 | 80.9 |

The results in Table 11 clearly show that there are marine oils with a low Lipid Prevention Score such as the oil extracted from squid and cod's liver. These may be used individually or mixed with vegetable oils in order to optimize the LPS of most marine oils with high score.

1-9. (canceled)

10. A food supplement comprising
a first oil having a first lipid prevention score higher than the total lipid content in this first oil;
a second oil having a second lipid prevention score lower than the total lipid content in this second oil;
said food supplement characterized in that the sum of the first lipid prevention score and the second lipid prevention score is substantially equal to the total liquid content in the food supplement.

11. The food supplement of claim 10, wherein the first oil comprises a marine oil.

12. The food supplement of claim 10, wherein the marine oil is chosen from the group including the extracted oil from the byproducts of fattened bluefin tuna, the krill oil, the mackerel oil, the sardine oil.

13. The food supplement of claim 10, wherein the extracted oil from the byproducts of fattened bluefin is chosen from the group including the oil extracted from mesentery, the oil extracted from liver, the oil extracted from swim bladder, the oil extracted from stomach, the oil extracted from heart, the oil extracted from kidneys.

14. The food supplement of claim 11, wherein the second oil comprises
a vegetable oil chosen from the group including nigella oil, corn oil, sweet almond oil, linseed oil, peanut oil, hazelnut oil, sunflower oil, olive oil, canola oil, nut oil; and/or
a marine oil chosen from the group including anchovy oil, squid oil, cod liver oil.

15. The food supplement of claim 10, wherein the olive oil is chosen from the group including Chetoui olive oil variety, Chemlali olive oil variety, Chemchali olive oil variety, Sayali olive oil variety, Meski olive oil variety, Zalmati olive oil variety, Oueslati olive oil variety.

16. The food supplement of claim 10, wherein
the first oil is an extracted oil from the byproducts of fattened bluefin, this extracted oil being chosen from the oil extracted from mesentery, the oil extracted from liver, the oil extracted from swim bladder, the oil extracted from stomach, the oil extracted from heart, the oil extracted from kidneys;
the proportion, in the food supplement, of the Chemlali olive oil variety is chosen from the group including
23,7 percent of the food supplement comprising oil extracted from mesentery;
26 percent of the food supplement comprising oil extracted from swim bladder;
16,7 percent of the food supplement comprising oil extracted from liver;
47,6 percent of the food supplement comprising oil extracted from liver;
41,4 percent of the food supplement comprising oil extracted from heart;
8,3 percent of the food supplement comprising oil extracted from kidney.

17. The food supplement of claim 10, further comprising anti-oxidizing agents, and/or anti-flavors agents, and/or colorants agents, and/or anti-aging agents.

18. The food supplement of claim 10, wherein it is in the form of capsules, or liquids, or liquid gels.

19. Method for balancing, in terms of fatty acids, a first amount of a first oil, this first oil having a first lipid prevention score higher than the total lipid content in this first oil, said method comprising
a selecting step of a second oil having a second lipid prevention score lower than the total lipid content in this second oil;
an adding step of a second amount of the second oil to the first amount of the first oil so that the sum of the first lipid prevention score of the first amount and the second lipid prevention score of the second amount is substantially equal to the total lipid content in the mixture of the first amount of the first oil and the second amount of the second oil.

20. The method of claim 19, wherein the first oil comprises a marine oil.

21. The method of claim 19, wherein the marine oil is chosen from the group including the extracted oil from the byproducts of fattened bluefin tuna, the krill oil, the mackerel oil, the sardine oil.

22. The method of claim 19, wherein the extracted oil from the byproducts of fattened bluefin is chosen from the group including the oil extracted from mesentery, the oil extracted from liver, the oil extracted from swim bladder, the oil extracted from stomach, the oil extracted from heart, the oil extracted from kidneys.

23. The method of claim 19, wherein the second oil comprises
a vegetable oil chosen from the group including nigella oil, corn oil, sweet almond oil, linseed oil, peanut oil, hazelnut oil, sunflower oil, olive oil, canola oil, nut oil; and/or
a marine oil chosen from the group including anchovy oil, squid oil, cod liver oil.

24. The method of claim 19, wherein the olive oil is chosen from the group including Chetoui olive oil variety, Chemlali olive oil variety, Chemchali olive oil variety, Sayali olive oil variety, Meski olive oil variety, Zalmati olive oil variety, Oueslati olive oil variety.

25. The method of claim 19, wherein
the first oil is an extracted oil from the byproducts of fattened bluefin, this extracted oil being chosen from the oil extracted from mesentery, the oil extracted from liver, the oil extracted from swim bladder, the oil extracted from stomach, the oil extracted from heart, the oil extracted from kidneys;
the second oil is Chemlali olive oil variety, the proportion, in the mixture of the first amount of the first oil and the second amount of the second oil, is chosen from the group including
23,7 percent of the food supplement comprising oil extracted from mesentery;
26 percent of the food supplement comprising oil extracted from swim bladder;
16,7 percent of the food supplement comprising oil extracted from liver;
47,6 percent of the food supplement comprising oil extracted from liver;
41,4 percent of the food supplement comprising oil extracted from heart;
8,3 percent of the food supplement comprising oil extracted from kidney.

26. The use of the food supplement of claim 10 to fight against hypercholesterolemia, this food supplement being applied locally, enterally or parenterally.

27. The use of the food supplement of claim 10 to fight against cardiovascular diseases, this food supplement being applied locally, enterally or parenterally.
